(19) United States
(12) Patent Application Publication
(10) Pub. No.: US 2014/0358136 A1
(43) Pub. Date: Dec. 4, 2014

(54) APPARATUS AND METHODS RELATED TO SELECTIVE THERMAL INSULATION OF CRYOGENIC BALLOONS FOR LIMITED CRYOGENIC ABLATION OF VESSEL WALLS

(71) Applicants: Brian KELLY, Ballybrit (IE); Gary Kelly, Windsor (IE); Barry Mullins, Wicklow (IE); Fiachra Sweeney, Ballybrit (IE)

(72) Inventors: Brian KELLY, Ballybrit (IE); Gary Kelly, Windsor (IE); Barry Mullins, Wicklow (IE); Fiachra Sweeney, Ballybrit (IE)

(21) Appl. No.: 14/114,367

(22) PCT Filed: Apr. 25, 2012
(86) PCT No.: PCT/US12/34910

§ 371 Date: Jun. 12, 2014

Related U.S. Application Data
(60) Provisional application No. 61/572,287, filed on Apr. 25, 2011.

(51) Int. Cl. A61B 18/02

(52) U.S. Cl. CPC A61B 18/02; A61B 2018/00345
USPC 606/21

(57) ABSTRACT

Embodiments related to cryogenically ablating a vessel wall in a partial circumferential, non-continuous, or helical ablation pattern are disclosed. A catheter is disclosed that includes a cryoballoon for ablation of the vessel wall. A radially expandable insulative element is disposed over the cryoballoon to shield non-targeted tissue of the vessel wall from the cryoballoon and prevent ablation of the non-targeted tissue. Partial circumferential, non-continuous, and helical ablation can be effective for treating a variety of renal, cardio-renal, and other diseases including but not limited to hypertension, heart failure, renal disease, renal failure, contrast nephropathy, arrhythmia, and myocardial infarction. The insulative element may be, for example, a sheath component having opening(s) formed therethrough or may be an outer balloon within which the cryoballoon is disposed.

### CROSS REFERENCE TO RELATED APPLICATION

This disclosure claims the benefit of U.S. Provisional Application No. 61/572,287, filed Apr. 25, 2011, which is incorporated herein by reference in its entirety.

### TECHNICAL FIELD

The present technology relates in general to cryotherapy, and in particular, to apparatus and methods for cryogenically cooling a targeted area of an inner surface of an anatomical vessel or other tissue.

### BACKGROUND

Cryotherapy can be a useful treatment modality in a wide range of catheter-based interventional procedures. For example, cryotherapeutic cooling can be used to modulate nerves or affect other tissue proximate anatomical vessels (e.g., blood vessels, other body lumens, or other areas in the body). This can reduce undesirable neural activity to achieve therapeutic benefits. Catheter-based neuromodulation utilizing cryotherapy can be used, for example, to modulate nerves and thereby reduce pain, local sympathetic activity, systemic sympathetic activity, associated pathologies, and other conditions. Furthermore, cryotherapy can be used, for example, for ablating tumors and treating stenosis. In some cryotherapeutic procedures, it can be useful to deliver cryotherapy via a balloon that can be expanded within an anatomical vessel. Such balloons can be operatively connected to extracorporeal support components (e.g., refrigerant supplies). As the applicability of cryotherapy for surgical intervention continues to expand, there is a need for innovation in the associated devices, systems, and methods. Such innovation has the potential to further expand the role of cryotherapy as a tool for improving the health of patients.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present technology.

FIG. 1 is a partially schematic isometric detail view of a common location of neural fibers proximate an artery.

FIG. 2 is a side view of a cryotherapy catheter having a cryoballoon at the distal end thereof, wherein the cryoballoon is in an expanded configuration.

FIG. 2A is a cross-sectional view taken along line A-A of FIG. 2.

FIG. 2B is a sectional view taken along line B-B of FIG. 2.

FIG. 2C is a cross-sectional view taken along line C-C of FIG. 2.

FIG. 3 is a side view of a distal portion of a sheath component disposed over the cryoballoon of FIG. 1 for shielding non-targeted tissue from the cryoballoon in accordance with an embodiment hereof.

FIG. 4 is a side view of a distal portion of a sheath component for shielding non-targeted tissue from a cryoballoon according to another embodiment hereof.

FIG. 5 is a side view of a dual balloon catheter having an insulative outer balloon with an inner cryoballoon, wherein the outer balloon shields non-targeted tissue from the cryoballoon.

FIG. 5A is a cross-sectional view taken along line A-A of FIG. 5.

FIG. 5B is a partially schematic cross-sectional view of an artery having the insulative outer balloon and inner cryoballoon of FIG. 5 deployed therein.

FIG. 6 is a side view of a distal portion of a dual balloon assembly according to another embodiment hereof.

FIG. 7 is a side view of a distal portion of a dual balloon assembly according to another embodiment hereof, wherein an inner cryoballoon has two expandable portions serially disposed along a length thereof.

FIG. 8 is a side view of a distal portion of a dual balloon assembly according to another embodiment hereof, wherein an inner cryoballoon has two expandable portions serially disposed along a length thereof with the expandable portions having inflated configurations that are radially offset from each other.

### DETAILED DESCRIPTION

Specific embodiments of the present technology are now described with reference to the figures, wherein like reference numbers indicate identical or functionally similar elements. The terms “distal” and “proximal” are used in the following description with respect to a position or direction relative to the treating clinician. “Distal” and “distally” refer to positions distant from or in a direction away from the clinician. “Proximal” and “proximally” refer to positions near or in a direction toward the clinician.

The following detailed description discloses specific examples of the technology, but it is not intended to limit the present technology or the application and uses of the present technology. For example, although the description discloses the present technology in the context of treatment of blood vessels, such as the coronary, carotid, and renal arteries, the present technology may also be used in any other body passageways or tissues where it is deemed useful. Furthermore, there is no intention to be bound by any expressed or implied theory presented herein.

In recent years, ablation of tissue has been used to modulate neural fibers that contribute to renal function. Ablation may be accomplished in various ways, including delivery of radio frequency (RF) energy, other suitable heating energies, or cryotherapy. Modulation of renal nerves is expected to be useful in treating a variety of renal, cardio-renal, and other diseases including heart failure, renal disease, renal failure, hypertension, contrast nephropathy, arrhythmia, and myocardial infarction. Furthermore, renal neuromodulation is expected to reduce renal sympathetic nervous activity, which can increase removal of water and sodium from the body and return renin secretion to more normal levels. Normalized renin secretion can cause blood vessels supplying the kidneys to assume a steady state level of dilation and constriction corresponding to adequate renal blood flow.

In neuromodulation procedures, it may be desirable to perform circumferential ablation that extends continuously about a full 360° of the circumference of an anatomical vessel to positively affect a medical condition. For example, in the treatment of atrial fibrillation or other arrhythmia, a circumferential treatment may be achieved by forming a circumferential lesion that is continuous completely about a normal cross-section of the pulmonary vein to disrupt aberrant electrical signals. In the treatment of heart failure, a circumferential treatment may be achieved by forming a similar continuous circumferential lesion that is continuous completely about a normal cross-section of a renal artery to reduce renal sympathetic neural activity. However, in some cases, it can be desirable to reduce structural changes to a blood vessel and avoid a circumferential ablation lesion along a single radial plane or cross-section of a blood vessel. Partial circumferential, non-continuous, or helical ablation are expected to be effective to treat a variety of renal, cardio-renal, and other diseases including those listed herein with less structural changes to vessels than fully circumferential, continuous, and non-helical ablation.

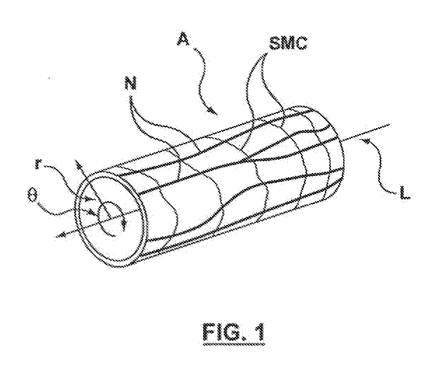

FIG. 1 illustrates a common anatomical arrangement of neural structures relative to body lumens or vascular structures, typically arteries. Neural fibers N generally may extend longitudinally along a lengthwise or longitudinal dimension L of an artery A about a relatively small range of positions along the radial dimension r, often within the adventitia of the artery. The artery A has smooth muscle cells SMC that surround the arterial circumference and generally spiral around the angular dimension θ of the artery, also within a relatively small range of positions along the radial dimension r. The smooth muscle cells SMC of the artery A accordingly have a lengthwise or longer dimension generally extending transverse (i.e., non-parallel) to the lengthwise dimension of the blood vessel.

Neuromodulation may be accomplished by ablating tissue through the use of an ablation catheter. As utilized herein, the term ablation includes the creation of scar tissue or a lesion that blocks or disrupts nerve conduction. In embodiments hereof, freezing temperatures or cryotherapy can be utilized to thermally damage or ablate target tissue of an artery to achieve neuromodulation of the target neural fibers. As compared to ablation lesions formed via radiofrequency energy, cryotherapy typically utilizes much less power to achieve neuromodulation.

Some embodiments hereof are related to protecting non-target tissue from cryogenic ablation by a cryotherapy catheter in order to produce a partial circumferential, non-continuous, or helical ablation lesion. As described above, partial circumferential, non-continuous, or helical ablation may be desirable in some cases. Partial circumferential, non-continuous, or helical ablation of a vessel can alter the sympathetic nervous system and can be effective for treating a variety of renal, cardio-renal, and other diseases including but not limited to hypertension, heart failure, renal disease, renal failure, contrast nephropathy, arrhythmia, and myocardial infarction. In order to form a partial circumferential, non-continuous, or helical ablation lesion, the cryotherapy from a cryoballoon can be focused on or limited to a targeted region of tissue to be treated and non-targeted tissue can be protected from ablation by an insulative element utilized in conjunction with the cryotherapy catheter that protects or shields non-targeted tissue from ablation using the various apparatus and methods described herein. As will be explained in more detail herein, the insulative element may be a sheath component having one or more openings formed therethrough, an insulative balloon disposed over or within the cryoballoon, or another suitable structure.

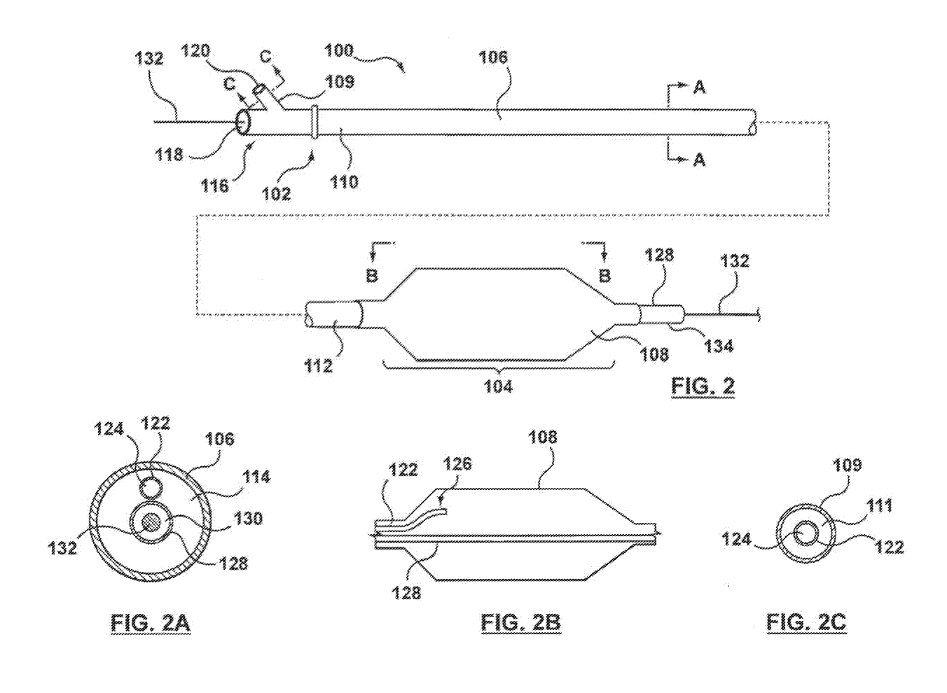

FIGS. 2, 2A, 2B, and 2C illustrate a cryotherapy balloon catheter 100 for use with an insulative element as described herein. Cryotherapy balloon catheter 100 can be utilized for ablating tissue to modulate targeted nerves. Cryotherapy catheter 100 includes a proximal portion 102 that extends out of the patient and has a hub 116. Distal portion 104 of catheter 100 is positionable at a targeted location within the vasculature and includes a cryoballoon 108, which is shown in an expanded or inflated configuration in FIG. 2. In the embodiment shown in FIGS. 2 and 2A, catheter 100 has an over-the-wire (OTW) catheter configuration with an inner shaft 128 that defines a guidewire lumen 130 extending substantially the entire length of the catheter for accommodating a guidewire 132. Inner shaft 128 has a proximal end (not shown) coupled to a proximal guidewire port 118 of hub 116 and a distal end 134 terminating distally of cryoballoon 108 and defining a distal guidewire port. Catheter 100 also includes a tubular component or outer shaft 106 which defines a lumen 114 and has a proximal end 110 coupled to hub 116 and a distal end 112 coupled to cryoballoon 108.

Catheter 100 further includes a cryo-supply shaft 122 extending through outer shaft 106. The cryo-supply shaft 122 defines an inflation lumen 124 and has a proximal end (not shown) coupled to hub 116 and a distal end 126 (see FIG. 2B) that terminates within cryoballoon 108. A cryo-inflation port 120 of hub 116 is in fluid communication with inflation lumen 124 of cryo-supply shaft 122. Cryo-supply shaft 122 receives and delivers a cryogenic agent such as N₂O liquid into cryoballoon 108 at a high pressure, e.g., 800 psi, such that there is a pressure drop when the cryogenic agent enters the interior of cryoballoon 108 and expands to a gas. The cryogenic agent may be any liquid having a boiling point lower than approximately −10° C. at atmospheric pressure, such as but not limited to N₂O liquid or CO₂ liquid. During the phase change of the cryogenic agent, a cooling effect takes place because expansion of compressed gas is an endothermic process that absorbs energy in the form of heat and thus results in cooling of the surroundings. Accordingly, as the cryogenic agent expands into gas, cryoballoon 108 is expanded or inflated and the exterior surface of the cryoballoon is cooled to cryogenic temperatures operable to ablate or thermally damage tissue. The temperature of cryoballoon 108 can be approximately between −5° C. and −120° C., which can result in modulation of neural fibers located adjacent to cryoballoon 108. As would be understood by one of ordinary skill in the art of balloon catheter design, hub 116 can provide a luer hub or other type of fitting that may be connected to a source of the cryogenic agent and may be of another construction or configuration without departing from the scope of the present technology.

As shown in the sectional view of FIG. 2B, cryo-supply shaft 122 and inner shaft 128 extend freely through, e.g., are not bonded to, outer shaft 106 and cryoballoon 108. As noted above, a continuous supply of cryofluid exits distal end 126 of cryo-supply shaft 122 into an interior of cryoballoon 108 to expand therein. Concurrently, the expanded cryogenic gas proximally exits the interior of cryoballoon 108 via a space between shafts 122, 128 and outer shaft 106. In an embodiment, a vacuum may be utilized to pull the expanded cryogenic gas out of the catheter although the vacuum is not required for the gas to exit. The expanded cryogenic gas travels proximally within lumen 114 of outer shaft 106 to exit or exhaust from catheter 100 via an arm 109 of hub 116. As shown in the cross-sectional view of FIG. 2C, cryotherapy shaft 122 is configured to extend through arm 109 such that an exhaust space 111 is defined between cryo-supply shaft 122 and an inner surface of arm 109 through which the expanded cryogenic gas may escape.

The multiple catheter shafts of catheter 100, e.g., outer shaft 106, inner shaft 128, and cryo-supply shaft 122, may be formed of a polymeric material, non-exhaustive examples of which include polyethylene, polyethylene block amide copolymer (PEBA), polyamide, and/or combinations thereof, which can be laminated, blended, co-extruded, or processed according to another suitable method. In an embodiment, inner shaft 128 may be a flexible tube of a polymeric material, such as, e.g., polyethylene tubing. Optionally, outer shaft 106 or some portion thereof may be formed as a composite having a reinforcement material incorporated within a polymeric body in order to enhance strength and/or flexibility. Suitable reinforcement layers can include braiding, wire mesh layers, embedded axial wires, embedded helical or circumferential wires, and the like. In one embodiment, for example, at least a proximal portion of outer shaft 106 may be formed from a reinforced polymeric tube. In addition, although catheter 100 is described herein as being constructed with various shafts extending therethrough for forming lumens of the catheter, it will be understood by those of ordinary skill in the art that other types of catheter construction are also possible, such as, without limitation thereto, a catheter shaft formed by multi-lumen profile extrusion. In another embodiment, catheter 100 may be modified to be of a rapid exchange (RX) catheter configuration without departing from the scope of the present technology such that inner shaft 128 extends within only the distal portion of catheter 100.

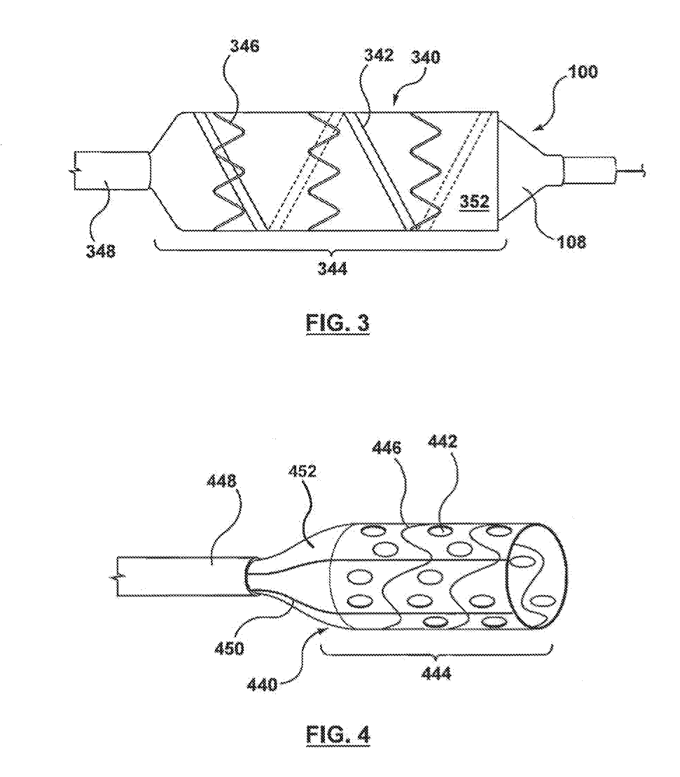

As previously mentioned, in order to form a partial circumferential, non-continuous, or helical ablation lesion, an insulative element can be utilized in conjunction with cryotherapy catheter 100 to protect or shield non-targeted tissue from ablation. In one embodiment hereof, the insulative element is an insulative sheath that may be disposed over the cryoballoon for shielding non-targeted tissue from the cryoballoon. More particularly, referring to FIG. 3, a distally located sheath component 344 of a sheath assembly 340 is shown distally extended from a guide catheter 348 to be disposed over cryoballoon 108 of catheter 100. Sheath component 344 is a tubular component defining a lumen having a length for receiving at least a working length of cryoballoon 108 of cryotherapy catheter 100. The working length of the balloon as used herein is intended to describe the longitudinal portion of the balloon which expands against and contacts the vessel wall. A proximal portion (not shown) of sheath assembly 340 extends through guide catheter 348 out of a patient such that it can be manipulated by a clinician. In an embodiment, sheath assembly 340 is an elongated tubular component slidingly disposed over catheter 100. The tubular component can have a distal end that is sheath component 344 and a proximal end that is coupled to a handle (not shown) to allow an operator to grip, push, and/or pull sheath assembly 340.

The sheath component 344 of sheath assembly 340 can be formed of an insulative material 352 effective for shielding or blocking ablation of tissue from cryoballoon 108, such as but not limited to nylon, polyurethane, PEBAX polymer, or silicone. Sheath component 344 includes one or more opening(s) 342 formed through insulative material 352 thereof. The sheath component 344 can be molded, extruded, or formed in another suitable manner. Furthermore, the opening(s) 342 can be formed, for example, using laser cutting or another suitable technique. Tissue of the vessel wall can come into contact or near-contact with cryoballoon 108 through opening(s) 342. Accordingly, opening(s) 342 can serve as areas for cryotherapy ablation and the geometry of opening(s) 342 therefore can form an ablation therapy pattern. In other embodiments, the opening(s) 342 can be replaced with other features that are relatively thermally transmissive (e.g., relatively thin or relatively thermally conductive portions of the sheath component 344). In FIG. 3, opening(s) 342 form a continuous spiral or corkscrew shape about a circumference of sheath component 344 that results in a helical ablation pattern. It will be apparent to those of ordinary skill in the art that other configurations are possible. For example, as shown in an alternative configuration in FIG. 4, openings 442 are depicted as a plurality of circular holes. In another embodiment, the openings may include a pattern of one or more longitudinal strips, one or more arcs extending around a portion of the circumference of the sheath component, or another pattern that can cause tissue adjacent to the pattern to be ablated to form a partial-circumferential, non-continuous, or helical lesion.

In an embodiment, in order to deploy sheath component 344 into contact with the vessel wall at the treatment site, a plurality of radially compressible annular supports or stents 346 are coupled to a surface of insulative material 352. Although depicted with three annular supports 346 approximately equally spaced along sheath component 344, it will be understood by those of ordinary skill in the art that any number of annular supports 346 may be utilized for radially expanding sheath component 344 and that the spacing therebetween may vary according to the intended application. Each annular support 346 is formed from a self-expanding spring member that is deployed upon release from a restraining mechanism, such as guide catheter 348. For example, annular supports 346 may be constructed of a superelastic material such as nitinol. Annular supports 346 may be attached or mechanically coupled to insulative material 352 of sheath component 344 by adhesive, welding, or bonding onto either an interior or exterior surface of insulative material 352. Annular supports 346 may have any suitable configuration, such as wavelike or sinusoidal patterned wire rings, a series of connected compressible diamond structures or other compressible spring members biased in a radially outward direction, that when released, bias sheath component 344 into conforming fixed engagement with an interior surface of the vessel wall. Examples of such annular support structures are described, for example, in U.S. Pat. No. 5,713,917 and U.S. Pat. No. 5,824,041, which are incorporated by reference herein in their entirety.

In use, sheath component 344 can be distally advanced out of a lumen of guide catheter 348 to a treatment site within a vessel. When released from guide catheter 348, annular supports 346 radially expand to bring an outer surface of insulative material 352 of sheath component 344 into contact with the interior surface the vessel wall. After sheath component 344 is deployed at the treatment site, catheter 100 is distally advanced in order to place cryoballoon 108 within sheath component 344 at the treatment site. Cryoballoon 108 is then expanded within sheath component 344 such that targeted tissue at the treatment site comes into contact or near-contact with cryoballoon 108 through opening(s) 342, which results in partial circumferential, non-continuous, or helical ablation of targeted tissue of the vessel wall. In areas in which insulative material 352 of sheath component 344 is positioned between cryoballoon 108 and the vessel wall, the non-targeted tissue is protected and shielded from ablation. After ablation of targeted tissue is complete, cryoballoon 108 is deflated and catheter 100 is subsequently withdrawn. Sheath component 344 is proximally withdrawn into guide catheter 348, which radially compresses annular supports 346 to allow for removal of sheath assembly 340.

In an embodiment, the sheath component may additionally include longitudinally-extending support struts to improve retraction of the sheath component into the guide catheter after the ablation procedure is complete. More particularly, as shown in FIG. 4, sheath component 444 of sheath assembly 440 includes a plurality of longitudinally-extending support struts 450 in addition to annular supports 446. Struts 450 are generally straight segments or wires formed from a suitable material including but not limited to stainless steel, Nitinol, or rigid polymers such as PEEK or polyamide. Struts 450 may be attached or mechanically coupled to insulative material 452 of sheath component 444 by adhesive, welding, or bonding onto either the interior or exterior surface of insulative material 452. When sheath component 444 is proximally retracted into a lumen of guide catheter 448 for removal from the vasculature, longitudinal struts 450 assist in radially compressing sheath component 444 for ease of insertion into guide catheter 448.

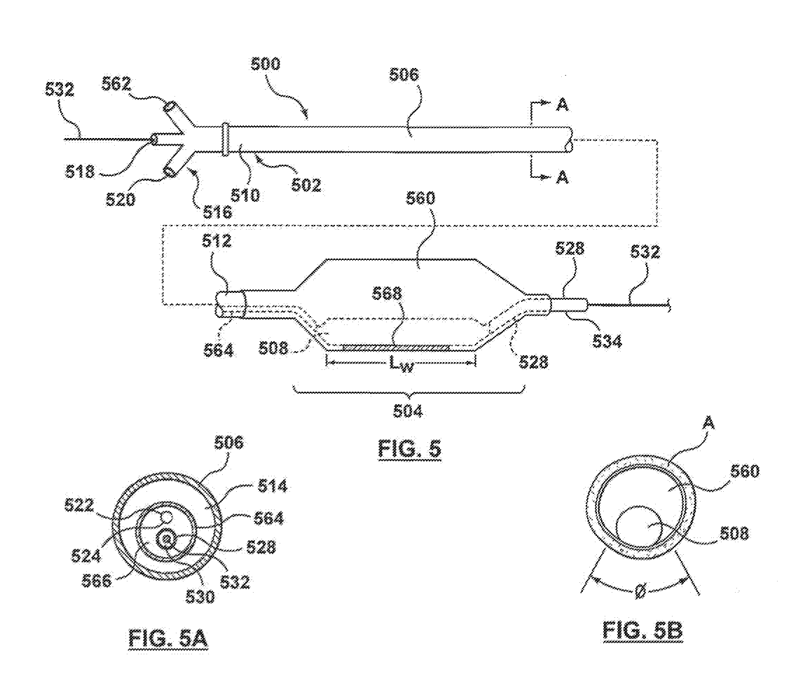

In another embodiment hereof, an insulative element for shielding non-targeted tissue from ablation is a second balloon disposed within or over cryoballoon 108. The inflation medium, e.g., air or saline, within the second balloon can provide insulation to adjacent tissue and prevent or block an exterior surface of the cryoballoon from coming into contact with non-targeted tissue. Referring to FIG. 5, a cryotherapy catheter 500 is utilized for ablating tissue to provide neuromodulation of the targeted nerves. As opposed to an insulative sheath for protecting non-targeted tissue from ablation, catheter 500 includes an insulative outer balloon 560 disposed over an inner cryoballoon 508 for shielding non-targeted tissue from ablation. In some embodiments, this portion of catheter 500 may be assembled by forming inner cryoballoon 508 in a folded configuration and inserting folded inner cryoballoon 508 into larger outer balloon 560.

Cryotherapy catheter 500 can include a proximal portion 502 that extends out of the patient and has a hub 516. Distal portion 504 of catheter 500 is positionable at a targeted location within the vasculature and includes outer balloon 560 and inner cryoballoon 508, which are both shown expanded or inflated in FIG. 5. Catheter 500 includes an outer shaft 506 which has a proximal end 510 coupled to hub 516 and a distal end 512 coupled to outer balloon 560. An inner tubular component or shaft 564 extends within lumen 514 of outer shaft 506 with a guidewire shaft 528 and cryo-supply shaft 522 extending through a lumen 566 defined by shaft 564. Cryoballoon 508 is positioned at the distal end of shaft 564 within an interior of outer balloon 560. Similar to inner shaft 128 described above, guidewire shaft 528 defines a guidewire lumen 530 extending substantially an entire length of catheter 500 for accommodating a guidewire 532. Guidewire shaft 528 has a proximal end (not shown) coupled to a proximal guidewire port 518 of hub 516 and a distal end 534 terminating distally of outer balloon 560 and defining a distal guidewire port. Cryo-supply shaft 522 defines an inflation lumen 524 and has a distal end (not shown) that terminates within cryoballoon 508. A cryo-inflation port 520 of hub 516 is in fluid communication with inflation lumen 524 of cryo-supply shaft 522, and cryo-supply shaft 522 receives and delivers a cryogenic agent such as N₂O liquid into cryoballoon 508 as described above with respect to cryosupply shaft 122 and cryoballoon 108.

The distal end of outer balloon 560 is coupled to guidewire shaft 528, and outer balloon 560 is inflated via an inflation medium delivered through a second inflation lumen 514. In an embodiment, inflation lumen 514 is defined between an inner surface of outer shaft 506 and an outer surface of inner shaft 564. Hub 516 includes a second inflation port 562 in fluid communication with second inflation lumen 514 for receiving an inflation medium, such as air or saline. As would be understood by one of ordinary skill in the art of balloon catheter design, hub 516 can provide a luer hub or other type of fitting that may be connected to sources of an inflation fluid and a cryogenic agent and may be of another construction or configuration without departing from the scope of the present technology.

Cryoballoon 508 can have a smaller expanded outer diameter than outer balloon 560. To achieve different expanded outer diameters, the balloons may be formed of materials having different compliances. Dilatation balloons may be classified, for example, as being compliant, noncompliant, or semi-compliant. Compliant balloons can be characterized by their ability to radially expand beyond their nominal diameters in response to increasing inflation pressure. Such balloons can be said to follow a stress-strain curve obtained by plotting balloon diameter versus inflation pressure. Noncompliant balloons can be characterized by nearly flat stress-strain curves illustrating that the balloon diameters expand relatively little over the range of usable inflation pressures. To achieve a smaller expanded outer diameter, cryoballoon 508 may be semi-compliant or non-compliant. In some embodiments, cryoballoon 508 can be 10% or less compliant and formed from PEBAX or nylon. Outer balloon 560 may be, for example, between 50% and 100% compliant and formed from polyurethane or silicone. Percentage compliance can correspond to the percentage of expansion that occurs between the cryoballoon 508 at an operating pressure and the cryoballoon 508 at a rated pressure (e.g., a burst pressure or a maximum inflation pressure). The recited values for percentage compliance can also apply to distensibility, which can be calculated as follows:

$$Distensibility = [ \frac{Diameter of Balloon at Selected Pressure}{Nominal Diameter of Balloon} - 1 ] \times 100 \%$$

The selected pressure can be an arbitrary, relatively high pressure (e.g., 10 bar). Suitable materials that may be utilized to achieve a desired amount of compliance for the balloons include but are not limited to polymers such as polyethylene, PEBA, PEBAX, nylon, silicone, polyethylene terephthalate (PET), polyamide, polyurethane, and copolymers or blends thereof.

A portion of the outer surface of cryoballoon 508 can be coupled to an interior surface of outer balloon 560. In an embodiment shown in FIG. 5, cryoballoon 508 is coupled to outer balloon 560 via a bond 568. In embodiments hereof, bond 568 may be formed, for example, with an adhesive, heat-bond, or weld. Forming the bond 568 can include expanding the cryoballoon 508 and attaching the outer balloon 560 to the expanded cryoballoon 508. Adhesive can be applied to an inside surface of the outer balloon 560 (e.g., using a needle extended through a distal neck of the outer balloon 560) prior to expanding the cryoballoon 508 and/or prior to introducing the cryoballoon 508 into the outer balloon 560. Furthermore, the adhesive can be a two-part adhesive (e.g., a two-part epoxy adhesive) and the two parts can be introduced onto the inner surface of the outer balloon 560 and the outer surface of the cryoballoon 508, respectively. The inner surface of the outer balloon 560 and the outer surface of the cryoballoon 508 can then be pressed together to combine the two parts, thereby activating the adhesive to form the bond 568. In use, outer balloon 560 expands into full circumferential contact with the vessel wall, and cryoballoon 508, which is attached to the interior surface thereof and has a smaller expanded outer diameter relative thereto, is essentially radially pulled toward only the portion of the vessel wall adjacent to where cryoballoon 508 is attached to outer balloon 560. When the cryogenic agent is introduced into cryoballoon 508, non-targeted tissue that is not adjacent to cryoballoon 508 is shielded or protected from ablation by the inflation medium located within outer balloon 560, which effectively acts as insulation.

Targeted tissue adjacent to cryoballoon 508 is ablated, resulting in a partial circumferential, non-continuous, or helical ablation pattern or lesion. With reference to FIG. 5B, which is a cross-sectional view of an artery A having outer insulative balloon 560 and inner cryoballoon 508 of FIG. 5 deployed therein, an area of contact between the portion of the exterior surface of outer balloon 560 that substantially corresponds to where the expanded cryoballoon 508 contacts the interior surface of outer balloon 560 and the vessel wall may be considered a nominal treatment area. The nominal treatment area may be equal to or slightly smaller than the ablation pattern resulting from cryoballoon 508 because the ablation therapy may extend slightly beyond the borders of the nominal treatment area. For example, in one embodiment, the nominal treatment area of the cryoballoon may extend around between 45° and 225° of the vessel wall circumference while the resulting ablation pattern of the cryoballoon may extend around between 10° and 340° of the vessel wall circumference. However, for purposes of the present disclosure, the nominal treatment area and the ablation pattern are considered to be approximately equal. The nominal treatment area/ablation pattern depends upon both an ablation arc Ø of the cryoballoon and a working length LW of the cryoballoon. More particularly, the nominal treatment area/ablation pattern may be calculated by multiplying the length of the ablation arc LØ by the working length LW of cryoballoon 508. The expanded diameter of cryoballoon 508 determines the ablation arc Ø and therefore determines the amount of circumferential tissue cryogenically ablated. The length of ablation arc Ø may be roughly calculated by the equation LØ=R((2̂)/360), wherein R is the radius and Ø is the contact surface arc. As previously mentioned, in embodiments hereof, due to outer balloon 560 which shields non-targeted tissue the ablation arc Ø of cryoballoon 508 can be constrained or limited to a partial circumferential, non-continuous, or helical portion of the vessel wall.

In addition to shielding non-targeted tissue from ablation, in one embodiment outer balloon 560 also serves to moderate the temperature of the cryotherapy. For example, when N₂O liquid is utilized as the cryogenic agent, the phase change of the cryogenic agent to gas may result in a cryoballoon temperature in the range of −70° C. to −80° C. However, neuromodulation may be accomplished at temperatures between −10° C. and −40° C., and these higher temperatures may be preferred in certain applications to minimize unnecessary structural changes to the vessel. Since cryoballoon 508 expands within outer balloon 560 when each are deployed in a vessel during treatment, heat transfer occurs therebetween. Due to heat transfer from cryoballoon 508, an inflation fluid such as water or saline within outer balloon 560 may freeze but the decrease in resulting temperature of outer balloon 560 will not be to such an extent that thermal injury will occur. Thermal injury or neuromodulation generally occurs at temperatures below −5° C., while a frozen outer balloon 560 can have a temperature at or above −3° C. Notably, heat transfer between outer balloon 560 and cryoballoon 508 may be beneficial to increase the temperature of the cryogenically-cooled balloon outer surface from, e.g., −80° C., to a preferred temperature for ablation, e.g., between −10° C. and −40° C. Thus, the heat transfer between the balloons helps to moderate the temperature of the cryotherapy.

Turning now to FIG. 6, an inner cryoballoon and an insulative outer balloon according to another embodiment hereof is shown. In FIG. 6, rather than or in addition to utilizing a bond to couple the inner balloon to the outer balloon, inner cryoballoon 608 can be positioned against the interior surface of outer balloon 660 due at least in part to the shapes of proximal and distal cones of the balloons. The outer balloon can have a first side 670 and a second side 672. First portions 670A, 670B of proximal and distal cones 671A, 671B, respectively, can be along the first side 670, and radially opposing second portions 672A. 672B of proximal and distal cones 671A, 671B, respectively, can be along the second side 672. First portions 670A, 670B can be longer and steeper than second portions 672A, 672B, such that the second side 672 appears somewhat flattened relative to the first side 670. The steeper taper of first portions 670A, 670B can provide outer balloon 660 with a larger expanded diameter than cryoballoon 608 such that insulative inflation medium can be located adjacent to non-targeted tissue of the vessel wall. The relatively gradual tapers of second portions 672A, 672B can be approximately equal to symmetrical tapers of proximal and distal cones 676A, 676B of inner cryoballoon 608 such that the cryoballoon 608 lies generally flat against the interior surface of outer balloon 660. In addition to having approximately equal tapers or slopes, proximal and distal cones 676A, 676B of inner cryoballoon 608 can have lengths approximately equal to or slightly longer than second portions 672A, 672B of proximal and distal cones 671A, 671B in order to facilitate good wall contact between inner cryoballoon 608 and outer balloon 660. The non-symmetrical proximal and distal cones 671A, 671B of outer balloon 660 can assist in positioning inner cryoballoon 608 against the interior wall of outer balloon 660.

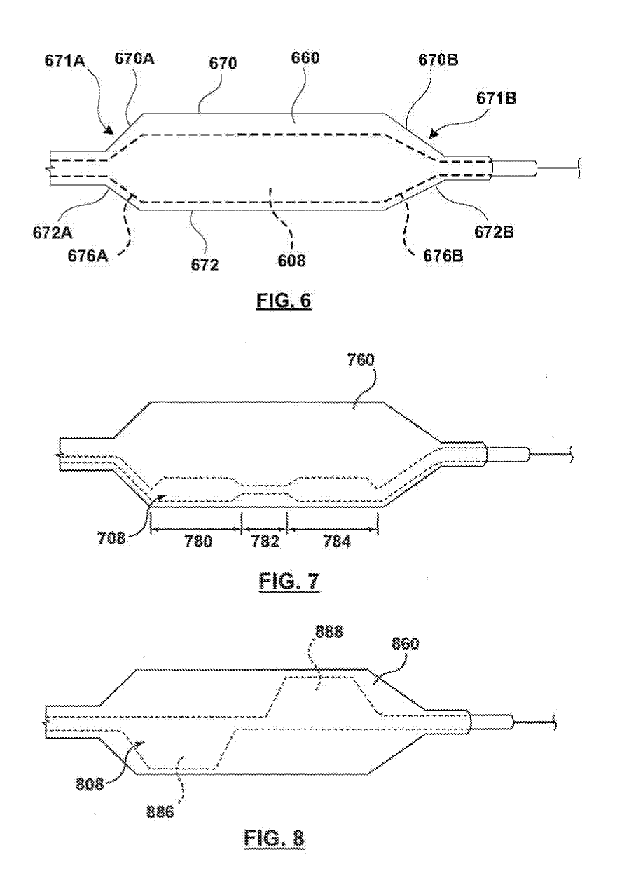

As previously noted, an expanded profile of the cryoballoon contributes to the ablation pattern. In embodiments hereof, the configuration or profile of the cryoballoon may be varied in order to achieve different ablation patterns within the vessel. FIG. 7 illustrates another configuration of an inner cryoballoon according to an embodiment hereof in which cryoballoon 708 includes two serially disposed expandable portions 780, 784 along a length thereof that are coupled to an interior surface of outer balloon 760. Expandable portions 780, 784 may be coupled to the interior surface of outer balloon 760 via a bond or adhesive (not shown). A constricted portion 782 is integrally formed between expandable portions 780, 784. Constricted portion 782 is of a smaller expanded outer diameter than expandable portions 780, 784 such that it does not contact the interior surface of outer balloon 760 and therefore is not adjacent to tissue of the vessel wall. Rather, insulative inflation medium such as air or saline from outer balloon 760 surrounds constricted portion 782 and protects the tissue adjacent to constricted portion 782 from ablation. This may cause only tissue adjacent to expandable portions 780, 784 to be ablated, resulting in an ablation pattern having two longitudinally spaced partial circumferential ablation lesions.

FIG. 8 illustrates another configuration of an inner cryoballoon according to an embodiment hereof in which cryoballoon 808 includes two serially disposed expandable portions 886, 888 having inflated configurations that are radially offset from each other and that are coupled to the interior surface of outer balloon 860. Expandable portions 886, 888 may be coupled to the interior surface of outer balloon 760 via a bond or adhesive (not shown). Tissue adjacent to expandable portions 886, 888 is ablated such that a non-continuous ablation pattern is formed having longitudinally spaced ablation lesions on radially opposing sides of the vessel wall.

In addition, although catheter 500 is described herein as being constructed with various shafts extending therethrough for forming lumens of the catheter, it will be understood by those of ordinary skill in the art that other types of catheter construction are also possible, such as, without limitation thereto, a catheter shaft formed by multi-lumen profile extrusion. Another possible modification of catheter 500 includes inner or guidewire shaft 528 extending through outer balloon 560 rather than cryoballoon 508.

As described herein, the cryoballoons of FIG. 5, FIG. 6, and FIG. 7 can be used to provide partial circumferential ablation of a vessel wall. However, in some applications, it may be desirable to perform full circumferential ablation of vessel walls that is also non-continuous or helical. Non-continuous, full circumferential ablation can include forming two or more partial circumferential ablations that collectively extend around the entire circumference of the vessel wall. Helical, full circumferential ablation can include forming one or more ablations that curve to extend around the entire circumference of the vessel wall without being fully circumferential in any single plane perpendicular to the vessel. The non-continuous or helical nature of these full circumferential ablations can reduce structural changes to any other region of the vessels in comparison to other full circumferential ablations. It will be understood by those of ordinary skill in the art that embodiments hereof for creating partial circumferential ablation patterns may also be utilized for creating non-continuous or helical full circumferential ablation patterns. For example, catheters having ablation assemblies which create partial circumferential ablation patterns may be longitudinally translated within a vessel and rotated as desired in order to perform multiple, sequential partial circumferential ablations which collectively extend around the entire circumference of the vessel wall. In some embodiments, relatively short balloons having lengths between 2 mm and 5 mm may be rotated and moved longitudinally in a vessel to produce a non-continuous and helical ablation pattern.

Since blood flow past a cryogenic balloon may affect the desired ablation pattern, embodiments described herein may include an occlusion balloon or other occlusive device. In the dual balloon embodiments described herein, the outer balloon can occlude blood flow when inflated against the vessel wall. In addition or alternatively, an occlusion balloon or other occlusive device may be placed proximal or distal to the outer balloon. Similarly, with respect to embodiments described in relation to FIG. 3 and FIG. 4, an occlusion balloon or other occlusive device may be placed proximal or distal to the cryoballoon. Suitable occlusive devices may be integrally formed on the delivery catheters or may be separate devices utilized in conjunction with the delivery catheters.

### Examples

1. A cryotherapeutic device, comprising:
an elongated shaft including a distal portion, the shaft configured to locate the distal portion in an anatomical vessel;
an elongated balloon at the distal portion;
a supply lumen along at least a portion of the shaft;
an exhaust lumen along at least a portion of the shaft, the exhaust lumen fluidly connected to the supply lumen via the balloon; and
a sheath at the distal portion configured radially expand, to receive at least a portion of the balloon, and to selectively expose a portion of a wall of the anatomical vessel to cryogenic cooling from the balloon, the portion of the wall of the anatomical vessel being non-circumferential in generally any plane perpendicular to a length of the balloon.

2. The cryotherapeutic device of example 1, wherein a distal end portion of the sheath is open.

3. The cryotherapeutic device of example 1, wherein the portion of the wall of the anatomical vessel is helical.

4. The cryotherapeutic device of example 1, wherein the sheath includes a cutout portion configured to expose the portion of the wall of the anatomical vessel to cryogenic cooling from the balloon.

5. The cryotherapeutic device of example 1, wherein the sheath includes a plurality of openings configured to expose the portion of the wall of the anatomical vessel to cryogenic cooling from the balloon.

6. The cryotherapeutic device of example 1, wherein the sheath includes at least one self-expanding annular support member.

7. The cryotherapeutic device of example 1, wherein the sheath includes at least one longitudinal strut.

8. A method for treating a patient, comprising:
locating a distal portion of an elongated shaft of a cryotherapeutic device within an anatomical vessel of the patient;
radially expanding a sheath within the anatomical vessel;
delivering refrigerant to a balloon of the cryotherapeutic device at the distal portion;
expanding the refrigerant within the balloon to cool the balloon;
radially expanding the balloon at least partially within the sheath; and
selectively exposing a portion of a wall of the anatomical vessel to cryogenic cooling from the balloon, the portion of the wall of the anatomical vessel being non-circumferential in generally any plane perpendicular to a length of the anatomical vessel.

9. The method of example 8, wherein selectively exposing the portion of the wall of the anatomical vessel includes cryogenically cooling the portion of the wall of the anatomical vessel through a cutout portion of the sheath.

10. The method of example 8, wherein—
the portion of the wall of the anatomical vessel is a first portion, and
selectively exposing the first portion includes—
   cryogenically cooling the first portion through a thermally transmissive portion of the sheath, and
   insulating a second portion of the wall of the anatomical vessel around the first portion from cryogenic cooling with a thermally insulative portion of the sheath.

11. The method of example 8, wherein radially expanding the sheath includes radially expanding at least one self-expanding annular support member of the sheath.

12. A cryotherapeutic device, comprising:
an elongated shaft including a distal portion, the shaft configured to locate the distal portion in an anatomical vessel;
a first balloon at the distal portion, the first balloon configured to expand into a first shape;
a supply lumen along at least a portion of the shaft;
an exhaust lumen along at least a portion of the shaft, the exhaust lumen fluidly connected to the supply lumen via the first balloon; and
a second balloon around the first balloon, the second balloon configured to expand into a second shape, wherein interaction between the first shape and the second shape causes the first balloon to locate preferentially in a radially offset position within the second balloon.

13. The cryotherapeutic device of example 12, wherein—
the second shape includes proximal and distal necks having greater slope in a first radial direction than in a second radial direction opposite to the first radial direction, and
the radially offset position is radially offset generally in the first radial direction.

14. The cryotherapeutic device of example 12, wherein the second shape includes proximal and distal necks that are non-symmetrical in a plane parallel to a length of the balloon.

15. The cryotherapeutic device of example 12, wherein—
the first balloon is non-compliant or semi-compliant, and
the second balloon is compliant.

16. A method for treating a patient, comprising:
locating a distal portion of an elongated shaft of a cryotherapeutic device within an anatomical vessel of the patient;
delivering refrigerant to a first balloon of the cryotherapeutic device at the distal portion;
expanding the refrigerant within the first balloon to cool the balloon;
radially expanding the first balloon into a first shape;
radially expanding a second balloon of the cryotherapeutic device at the distal portion around the first balloon into a second shape;
preferentially locating the first balloon in a radially offset position within the second balloon by interaction between the first shape and the second shape; and
cryogenically cooling a portion of a wall of the anatomical vessel, the portion being non-circumferential in generally any plane perpendicular to a length of the anatomical vessel.

17. The method of example 16, wherein—
the portion of the wall of the anatomical vessel is a first portion, and
the method further comprises insulating a second portion of the wall of the anatomical vessel around the first portion from cryogenic cooling with a space between the first balloon and a wall of the second balloon.

18. The method of example 16, wherein—
radially expanding the first balloon includes non-compliantly or semi-compliantly radially expanding the first balloon, and
radially expanding the second balloon includes compliantly expanding the second balloon.

19. The method of example 16, further comprising circulating a heat-transfer fluid through the second balloon to warm the first balloon and to reduce the cooling of the portion of the wall of the anatomical vessel.

20. The method of example 19, wherein circulating the heat-transfer fluid causes a temperature of the first balloon to be between −10° C. and −40° C.

### CONCLUSION

While various embodiments according to the present technology have been described above, it should be understood that they have been presented by way of illustration and example only, and not limitation. It will be apparent to persons skilled in the relevant art that various changes in form and detail can be made therein without departing from the spirit and scope of the present technology. Thus, the breadth and scope of the present technology should not be limited by any of the above-described embodiments. It will also be understood that each feature of each embodiment discussed herein, and of each reference cited herein, can be used in combination with the features of any other embodiment. All patents and publications discussed herein are incorporated by reference herein in their entirety.

Where the context permits, singular or plural terms may also include the plural or singular terms, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout the disclosure to mean including at least the recited feature(s) such that any greater number of the same feature(s) and/or additional types of other features are not precluded. It will also be appreciated that various modifications may be made to the described embodiments without deviating from the present technology. Further, while advantages associated with certain embodiments of the present technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

I/We claim:

1. A cryotherapeutic device, comprising:
an elongated shaft including a distal portion, the shaft configured to locate the distal portion in an anatomical vessel;
an elongated balloon at the distal portion;
a supply lumen along at least a portion of the shaft;
an exhaust lumen along at least a portion of the shaft, the exhaust lumen fluidly connected to the supply lumen via the balloon; and
a sheath at the distal portion configured radially expand, to receive at least a portion of the balloon, and to selectively expose a portion of a wall of the anatomical vessel to cryogenic cooling from the balloon, the portion of the wall of the anatomical vessel being non-circumferential in generally any plane perpendicular to a length of the balloon.

2. The cryotherapeutic device of claim 1, wherein a distal end portion of the sheath is open.

3. The cryotherapeutic device of claim 1, wherein the portion of the wall of the anatomical vessel is helical.

4. The cryotherapeutic device of claim 1, wherein the sheath includes a cutout portion configured to expose the portion of the wall of the anatomical vessel to cryogenic cooling from the balloon.

5. The cryotherapeutic device of claim 1, wherein the sheath includes a plurality of openings configured to expose the portion of the wall of the anatomical vessel to cryogenic cooling from the balloon.

6. The cryotherapeutic device of claim 1, wherein the sheath includes at least one self-expanding annular support member.

7. The cryotherapeutic device of claim 1, wherein the sheath includes at least one longitudinal strut.

8. A method for treating a patient, comprising:
locating a distal portion of an elongated shaft of a cryotherapeutic device within an anatomical vessel of the patient;
radially expanding a sheath within the anatomical vessel;
delivering refrigerant to a balloon of the cryotherapeutic device at the distal portion;
expanding the refrigerant within the balloon to cool the balloon;
radially expanding the balloon at least partially within the sheath; and
selectively exposing a portion of a wall of the anatomical vessel to cryogenic cooling from the balloon, the portion of the wall of the anatomical vessel being non-circumferential in generally any plane perpendicular to a length of the anatomical vessel.

9. The method of claim 8, wherein selectively exposing the portion of the wall of the anatomical vessel includes cryogenically cooling the portion of the wall of the anatomical vessel through a cutout portion of the sheath.

10. The method of claim 8, wherein—
the portion of the wall of the anatomical vessel is a first portion, and
selectively exposing the first portion includes—
cryogenically cooling the first portion through a thermally transmissive portion of the sheath, and
insulating a second portion of the wall of the anatomical vessel around the first portion from cryogenic cooling with a thermally insulative portion of the sheath.

11. The method of claim 8, wherein radially expanding the sheath includes radially expanding at least one self-expanding annular support member of the sheath.

12. A cryotherapeutic device, comprising:
an elongated shaft including a distal portion, the shaft configured to locate the distal portion in an anatomical vessel;
a first balloon at the distal portion, the first balloon configured to expand into a first shape;
a supply lumen along at least a portion of the shaft;
an exhaust lumen along at least a portion of the shaft, the exhaust lumen fluidly connected to the supply lumen via the first balloon; and
a second balloon around the first balloon, the second balloon configured to expand into a second shape, wherein interaction between the first shape and the second shape causes the first balloon to locate preferentially in a radially offset position within the second balloon.

13. The cryotherapeutic device of claim 12, wherein—
the second shape includes proximal and distal necks having greater slope in a first radial direction than in a second radial direction opposite to the first radial direction, and
the radially offset position is radially offset generally in the second radial direction.

14. The cryotherapeutic device of claim 12, wherein the second shape includes proximal and distal necks that are non-symmetrical in a plane parallel to a length of the balloon.

15. The cryotherapeutic device of claim 12, wherein—
the first balloon is non-compliant or semi-compliant, and
the second balloon is compliant.

16. A method for treating a patient, comprising:
locating a distal portion of an elongated shaft of a cryotherapeutic device within an anatomical vessel of the patient;
delivering refrigerant to a first balloon of the cryotherapeutic device at the distal portion;
expanding the refrigerant within the first balloon to cool the balloon;
radially expanding the first balloon into a first shape;
radially expanding a second balloon of the cryotherapeutic device at the distal portion around the first balloon into a second shape;
preferentially locating the first balloon in a radially offset position within the second balloon by interaction between the first shape and the second shape; and
cryogenically cooling a portion of a wall of the anatomical vessel, the portion being non-circumferential in generally any plane perpendicular to a length of the anatomical vessel.

17. The method of claim 16, wherein—
the portion of the wall of the anatomical vessel is a first portion, and
the method further comprises insulating a second portion of the wall of the anatomical vessel around the first portion from cryogenic cooling with a space between the first balloon and a wall of the second balloon.

18. The method of claim 16, wherein—
radially expanding the first balloon includes non-compliantly or semi-compliantly radially expanding the first balloon, and
radially expanding the second balloon includes compliantly expanding the second balloon.

19. The method of claim 16, further comprising circulating a heat-transfer fluid through the second balloon to warm the first balloon and to reduce the cooling of the portion of the wall of the anatomical vessel.

20. The method of claim 19, wherein circulating the heat-transfer fluid causes a temperature of the first balloon to be between −10° C. and −40° C.
